(19) United States
(12) Patent Application Publication
(10) Pub. No.: US 2024/0100314 A1
(43) Pub. Date: Mar. 28, 2024

(54) Connection Systems and Methods Thereof

(71) Applicant: Bard Access Systems, Inc., Salt Lake City, UT (US)

(72) Inventors: Kelton John Michael Stokes, Salt Lake City, UT (US); Stefan Josef Fellner, Salt Lake City, UT (US)

(73) Assignee: Bard Access Systems, Inc., Salt Lake City, UT (US)

(21) Appl. No.: 18/373,805

(22) Filed: Sep. 27, 2023

Related U.S. Application Data
(60) Provisional application No. 63/410,844, filed on Sep. 28, 2022.

(51) Int. Cl. A61M 39/10

(52) U.S. Cl. CPC A61M 39/1011; A61M 2039/1094; A61M 2207/10

(57) ABSTRACT

Connection systems and methods establish safe, routine, functional connections between sterile single patient-use (“SPU”) medical devices and multiple patient-use (“MPU”) medical devices. For example, a connection system can include a male connector associated with an SPU medical device and a female connector associated with an MTV medical device. The male connector can include a flexible member therearound but proximal of a plug of the male connector. The female connector can include a rigid member around an opening of a receptacle of the female connector. The flexible member can conform to the rigid member with a procedural barrier therebetween when the plug of the male connector is inserted into the receptacle of the female connector. Upon insertion of the plug of the male connector into the receptacle of the female connector, one or more functional connections can be established across the procedural barrier between the SPU and MPU medical devices.

### PRIORITY

This application claims the benefit of priority to U.S. Provisional Application No. 63/410,844, filed Sep. 28, 2022, which is incorporated by reference in its entirety into this application.

### BACKGROUND

Procedural fields are typically established about patients with one or more procedural barriers before any medical procedures. For example, sterile fields can be established over or around patients by covering the patients with sterile drapes. Oftentimes, the medical procedures require multiple patient-use (“MPU”) medical devices that cannot be sterilized, which mandate placement of such medical devices under the sterile drapes; however, sterile single patient-use (“SPU”) medical devices often need to be functionally connected to the MPU medical devices. Some existing solutions rely on arbitrarily breaching sterile drapes to make functional connections between sterile SPU medical devices and MPU medical devices. But arbitrarily breaching such sterile drapes risks contaminating the sterile fields carefully established about the patients. That, and establishing some functional connections between the sterile SPU medical devices and the MPU medical devices can be difficult, particularly through arbitrary, self-made breaches in the sterile drapes. What is needed is a connection system for establishing safe, routine, functional connections between sterile SPU medical devices and MPU medical devices. foregoing.

Disclosed herein are connection systems and methods that address the foregoing.

### SUMMARY

Disclosed herein is a connection system including, in some embodiments, a male connector associated with an SPU medical device and a female connector associated with an MPU medical device. The male connector includes a flexible member around the male connector, wherein the flexible member is proximal of a plug of the male connector. The female connector includes a rigid member around an opening of a receptacle of the female connector. The flexible member of the male connector is configured to conform to the rigid member of the female connector with a procedural barrier therebetween when the plug of the male connector is inserted into the receptacle of the female connector. Upon insertion of the plug of the male connector into the receptacle of the female connector, one or more functional connections are established across the procedural barrier between the SPU and MPU medical devices.

In some embodiments, the flexible member is fixedly coupled to the male connector.

In some embodiments, the flexible member is removably coupled to the male connector.

In some embodiments, the flexible member is a flexible disk.

In some embodiments, the rigid member is fixedly coupled to the female connector.

In some embodiments, the rigid member is removably coupled to the female connector.

In some embodiments, the rigid member is a rigid bowl opening away from the receptacle of the female connector.

In some embodiments, the male connector is magnetically secured to the female connector upon the insertion of the plug of the male connector into the receptacle of the female connector. The male connector is magnetically secured to the female connector via one or more magnets in each member of the flexible member and the rigid member.

In some embodiments, the male connector is mechanically secured to the female connector upon the insertion of the plug of the male connector into the receptacle of the female connector. The male connector is mechanically secured to the female connector via one or more retractable protrusions protruding from the plug of the male connector and one or more corresponding recesses in an inner wall of the receptacle.

In some embodiments, the one-or-more retractable protrusions and the one-or-more corresponding recesses produce an audible click-type sound upon the insertion of the plug of the male connector into the receptacle of the female connector. The audible click-type sound indicates the male connector is mechanically secured to the female connector.

In some embodiments, the male connector is adherently secured to the female connector upon the insertion of the plug of the male connector into the receptacle of the female connector. The male connector is adherently secured to the female connector via an adhesive over a mating surface of each member of the flexible and rigid member.

In some embodiments, conformation of the flexible member to the rigid member forms a fluid-tight seal.

In some embodiments, the one-or-more functional connections are selected from electrical, optical, and fluid connections.

In some embodiments, the procedural barrier is a sterile drape such that the one-or-more functional connections across the procedural barrier between the SPU and MPU medical devices extend from a sterile field including the SPU medical device and a non-sterile field including the MPU medical device.

Also disclosed herein is a method of a connection system including, in some embodiments, an inserting step. The inserting step includes inserting a male connector associated with an SPU medical device across a procedural barrier into a female connector associated with an MPU medical device. The male connector includes a flexible member around the male connector, wherein the flexible member is proximal of a plug of the male connector. The female connector includes a rigid member around an opening of a receptacle of the female connector. The flexible member is configured to conform to the rigid member with the procedural barrier therebetween. Upon insertion of the plug of the male connector into the receptacle of the female connector in the inserting step, one or more functional connections are established across the procedural barrier between the SPU and MPU medical devices.

In some embodiments, the flexible member is a flexible disk fixedly or removably coupled to the male connector.

In some embodiments, the rigid member is a rigid bowl fixedly or removably coupled to the female connector. The rigid bowl opens away from the receptacle of the female connector.

In some embodiments, the male connector is magnetically, mechanically, or adherently secured to the female connector upon the insertion of the plug of the male connector into the receptacle of the female connector in the inserting step. Conformation of the flexible member to the rigid member forms a fluid-tight seal.

In some embodiments, the one-or-more functional connections are selected from electrical, optical, and fluid connections.

In some embodiments, the procedural barrier is a sterile drape such that the one-or-more functional connections across the procedural barrier between the SPU and MPU medical devices extend from a sterile field including the SPU medical device and a non-sterile field including the MPU medical device.

These and other features of the concepts provided herein will become more apparent to those of skill in the art in view of the accompanying drawings and following description, which describe particular embodiments of such concepts in greater detail.

### DRAWINGS

FIG. 1 illustrates a connection system for establishing one or more functional connections across a procedural barrier between SPU and MPU medical devices in accordance with some embodiments.

FIG. 2 illustrates a detailed view of the connection system in accordance with some embodiments.

FIG. 3 illustrates longitudinal cross sections of the connection system before and after a plug of a male connector is inserted into a receptacle of a female connector in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a medical device includes a portion or section of the medical device intended to be near a clinician when the medical device is used on a patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device can include the proximal end of the medical device, and, in such instances, the proximal portion, the proximal section, or the proximal length of the medical device can be further specified as a “proximal-end portion,” a “proximal-end section,” or a “proximal-end length” of the medical device. That said, the proximal portion, the proximal section, or the proximal length of the medical device need not include the proximal end of the medical device. Indeed, unless context suggests otherwise, the proximal portion, the proximal section, or the proximal length of the medical device is not a terminal portion, terminal section, or terminal length of the medical device.

With respect to “distal,” a “distal portion” or “distal section” of, for example, a medical device includes a portion or section of the medical device intended to be near or in a patient when the medical device is used on the patient. Likewise, a “distal length” of the medical device includes a length of the medical device intended to be near or in the patient when the medical device is used on the patient. A “distal end” of the medical device is an end of the medical device intended to be near or in the patient when the medical device is used on the patient. The distal portion, the distal section, or the distal length of the medical device can include the distal end of the medical device, and, in such instances, the distal portion, the distal section, or the distal length of the medical device can be further specified as a “distal-end portion,” a “distal-end section,” or a “distal-end length” of the medical device. That said, the distal portion, the distal section, or the distal length of the medical device need not include the distal end of the medical device. Indeed, unless context suggests otherwise, the distal portion, the distal section, or the distal length of the medical device is not a terminal portion, terminal section, or terminal length of the medical device.

Unless defined otherwise, all technical and scientific terms used herein have the same meaning as commonly understood by those of ordinary skill in the art.

Again, procedural fields are typically established about patients with one or more procedural barriers before any medical procedures. For example, sterile fields can be established over or around patients by covering the patients with sterile drapes. Oftentimes, the medical procedures require MPU medical devices that cannot be sterilized, which mandate placement of such medical devices under the sterile drapes; however, sterile single patient-use SPU medical devices often need to be functionally connected to the MPU medical devices. Some existing solutions rely on arbitrarily breaching sterile drapes to make functional connections between sterile SPU medical devices and MPU medical devices. But arbitrarily breaching such sterile drapes risks contaminating the sterile fields carefully established about the patients. That, and establishing some functional connections between the sterile SPU medical devices and the MPU medical devices can be difficult, particularly through arbitrary, self-made breaches in the sterile drapes. What is needed is a connection system for establishing safe, routine, functional connections between sterile SPU medical devices and MPU medical devices.

Disclosed herein are connection systems and methods that address the foregoing. For example, a connection system can include a male connector associated with a SPU medical device and a female connector associated with a MPU medical device. The male connector can include a flexible member around the male connector but proximal of a plug of the male connector. The female connector can include a rigid member around an opening of a receptacle of the female connector. The flexible member can be configured to conform to the rigid member with a procedural barrier therebetween when the plug of the male connector is inserted into the receptacle of the female connector. Upon insertion of the plug of the male connector into the receptacle of the female connector, one or more functional connections can be established across the procedural barrier between the SPU and MPU medical devices.

### Connection Systems

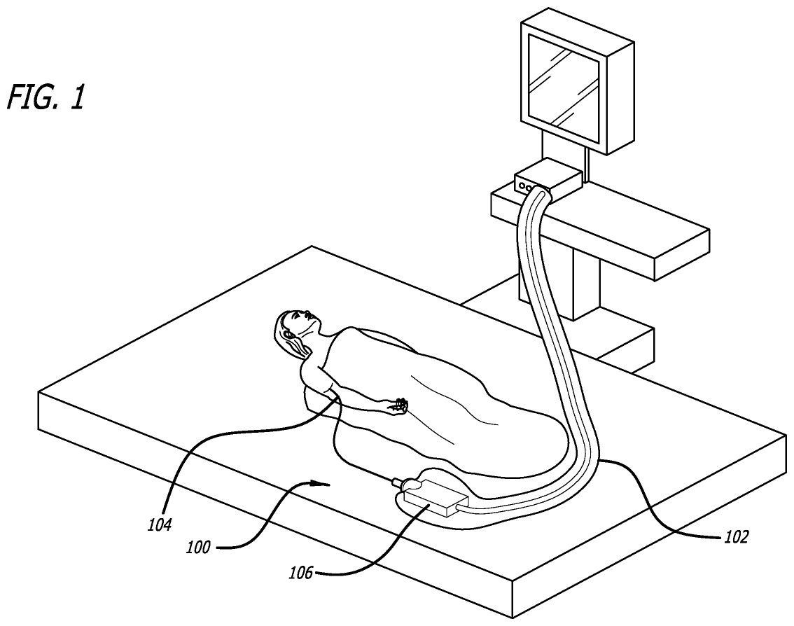

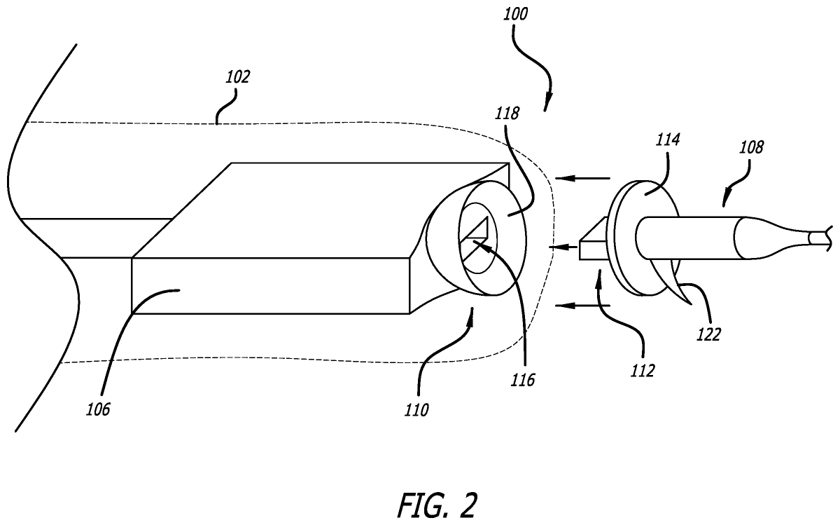

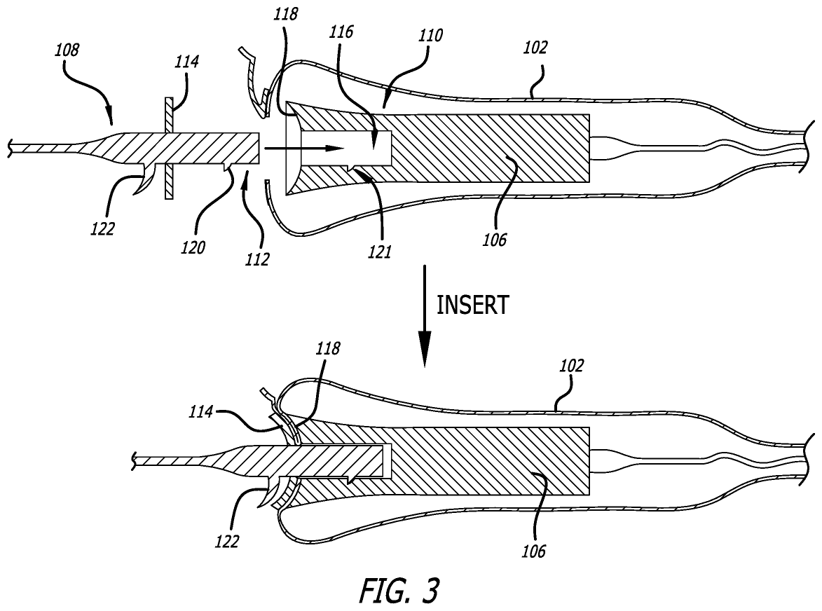

FIGS. 1-3 illustrate a connection system 100 for establishing one or more functional connections across a procedural barrier 102 between an SPU medical device 104 and an MPU medical device 106 in accordance with some embodiments.

As shown, the connection system 100 can include a male connector 108 associated with the SPU medical device 104 and a female connector 110 associated with the MPU medical device 106. As provided in further detail below, upon insertion of the male connector 108 into the female connector 110, one or more functional connections can be established across the procedural barrier 102 between the SPU and MPU medical devices 104 and 106, thereby maintaining an environment in which each medical device of the SPU and MPU medical devices 104 and 106 is located.

Notably, the foregoing or primary configuration of the connection system 100 can be reversed in a secondary configuration of the connection system 100 in which the male connector 108 is associated with the MPU medical device 106 and the female connector 110 is associated with the SPU medical device 104. However, the primary configuration of the connection system 100 is particularly advantageous when the procedural barrier 102 separates a sterile field including the SPU medical device 104 from a non-sterile field including the MPU medical device 106. This is because the primary configuration of the connection system 100 obviates having to breach the sterile field with a non-sterile component as necessitated by the secondary configuration of the connection system 100. Indeed, the secondary configuration of the connection system 100 necessitates breaching the sterile field including the female connector 110 of the SPU medical device 104 with the male connector 108 from the non-sterile field including the male connector 108 and the MPU medical device 106 in order to establish the one-or-more functional connections.

The male connector 108 can include a plug 112 and a flexible member 114 around the male connector 108 but proximal of the plug 112.

The plug 112 can be configured to insert into the receptacle 116 of the female connector 110. Such a plug 112 can be circular, rectangular, trapezoidal, or any other shape having C₁ or greater rotational symmetry in a transverse cross section of the plug 112. The plug 112 being circular in the transverse cross section thereof can be advantageous in that a clinician need not be concerned with a particular rotation of the plug 112 along its central axis to match the receptacle 116 of the female connector 110. As such, the plug 112 being circular in the transverse cross section thereof is generally associated with establishing a single functional connection in-line with the central axis of the plug 112 and a central axis of the receptacle 116. In contrast, the plug 112 being rectangular, trapezoidal, or the like in the transverse cross section thereof is generally associated with establishing two or more functional connections parallel to the central axes of the plug 112 and the receptacle 116 by enforcing a particular rotation of the plug 112 to match the receptacle 116 of the female connector 110.

The flexible member 114 can be fixedly coupled to the male connector 108, or the flexible member 114 can be removably coupled to the male connector 108. In addition, the flexible member 114 can be a flexible disk; however, it should be understood the flexible member 114 is not limited in shape to the disk. Indeed, whether the flexible member 114 has the shape of the disk or some other shape, the flexible member 114 can be configured to conform to the rigid member 118 of the female connector 110 with the procedural barrier 102 therebetween when the plug 112 is inserted into the receptacle 116. Notably, the flexible member 114 can also function as a guard to keep a clinician's male connector-holding hand proximal of the guard, thereby preventing the clinician's hand from crossing from a procedural field such as a sterile field into another field such as a non-sterile field.

The female connector 110 can include a receptacle 116 and a rigid member 118 around an opening of the receptacle 116.

The receptacle 116 of the female connector 110 can be configured to accept the plug 112 of the male connector 108. As such, the receptacle 116 can likewise be circular, rectangular, trapezoidal, or any other shape having C₁ or greater rotational symmetry in a transverse cross section of the receptacle 116.

The rigid member 118 can be fixedly coupled to the female connector 110, or the rigid member 118 can be removably coupled to the female connector 110. In addition, the rigid member 118 can be a rigid bowl opening away from the receptacle 116 of the female connector 110; however, it should be understood the rigid member 118 is not limited in shape to the bowl. Indeed, whether the rigid member 118 has the shape of the bowl, a dome, or some other shape, the rigid member 118 can be configured to support the flexible member 114 of the male connector 108 with the procedural barrier 102 therebetween when the plug 112 is inserted into the receptacle 116.

Notwithstanding the foregoing, it should be understood that the flexible member 114 and the rigid member 118 can be interchangeably coupled with either the male connector 108 or the female connector 110. Indeed, in an alternative of that set forth above, the flexible member 114 can be fixedly or removably coupled to the female connector 110, and the rigid member 118 can be fixedly or removably coupled to the male connector 108.

The male connector 108 and the female connector 110 can be mutually configured for magnetically securing the male connector 108 to the female connector 110, mechanically securing the male connector 108 to the female connector 110, or adherently securing the male connector 108 to the female connector 110 upon the insertion of the plug 112 of the male connector 108 into the receptacle 116 of the female connector 110. Combinations of the foregoing are also possible. Whether the male connector 108 and the female connector 110 are secured to each other in accordance with the foregoing upon the insertion of the plug 112 into the receptacle 116, and regardless of how the male connector 108 and the female connector 110 might be secured to each other, conformation of the flexible member 114 of the male connector 108 to the rigid member 118 of the female connector 110 can be configured to form a fluid-tight seal.

As to magnetically securing the male connector 108 to the female connector 110, the male connector 108 can be magnetically secured to the female connector 110 via one or more opposing magnets in each member of the flexible member 114 and the rigid member 118.

As to mechanically securing the male connector 108 to the female connector 110, the male connector 108 can be mechanically secured to the female connector 110 via one or more retractable protrusions 120 protruding from an outer wall of the plug 112 of the male connector 108 and one or more corresponding recesses 121 in an inner wall of the receptacle 116 of the female connector 110. Advantageously, the one-or-more retractable protrusions 120 and the one-or-more corresponding recesses 121 can be configured to produce an audible click-type sound upon the insertion of the plug 112 of the male connector 108 into the receptacle 116 of the female connector 110. Such an audible click-type sound can be used to indicate the male connector 108 is mechanically secured to the female connector 110. Notably, the one-or-more retractable protrusions 120 can be spring-loaded to protrude from the outer wall of the plug 112 for retraction by a lever 122 extending from the male connector 108 proximal of the retractable protrusions 120.

As to adherently securing the male connector 108 to the female connector 110, the male connector 108 can be adherently secured to the female connector 110 via an adhesive over a mating surface of one or both members of the flexible and rigid members 114 and 118. Additionally or alternatively, the adhesive can be around the plug-shaped hole on one or both sides of the plastic window of the procedural barrier 102 where it intervenes between the flexible and rigid members 114 and 118.

Upon insertion of the plug 112 of the male connector 108 into the receptacle 116 of the female connector 110, one or more functional connections can be established across the procedural barrier 102 between the SPU and MPU medical devices 104 and 106. The one-or-more functional connections are selected from electrical, optical, and fluid connections.

The procedural barrier 102 can be a sterile drape (e.g., a polypropylene drape) with an optional plug-sized hole in a plastic window of the sterile drape, wherein the plastic window, in turn, is set within a larger window-sized hole in the sterile drape for sealing the flexible and rigid members 114 and 118 thereagainst. Notably, the plug-shaped hole can be covered by an adhesive pull-tub prior to use of the sterile drape. The plastic window can be configured for placement between the flexible member 114 of the male connector 108 and the rigid member 118 of the female connector 110 with the plug-shaped hole centered over the receptacle 116 of the female connector 110. And, again, each side of the plastic window can include the adhesive thereon. The one-or-more functional connections can thusly be made across the sterile drape between the SPU and MPU medical devices 104 and 106 from a sterile field including the SPU medical device 104 and a non-sterile field including the MPU medical device 106.

### Methods

Methods of the connection system 100 include a method of using the connection system 100. Such a method includes at least an inserting step of inserting the male connector 108 associated with the SPU medical device 104 across the procedural barrier 102 into the female connector 110 associated with the MPU medical device 106. As set forth above, the male connector 108 can include the flexible member 114 around the male connector 108, wherein the flexible member 114 is proximal of the plug 112 of the male connector 108. The female connector 110 can include the rigid member 118 around the opening of the receptacle 116 of the female connector 110. Again, the flexible member 114 can be configured to conform to the rigid member 118 with the procedural barrier 102 therebetween. And, upon insertion of the plug 112 of the male connector 108 into the receptacle 116 of the female connector 110 in the inserting step, the one-or-more functional connections can be established across the procedural barrier 102 between the SPU and MPU medical devices 104 and 106.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A connection system, comprising:
a male connector associated with a single patient-use (“SPU”) medical device, the male connector including a flexible member around the male connector with the flexible member proximal of a plug of the male connector; and
a female connector associated with a multiple patient-use (“MPU”) medical device, the female connector including a rigid member around an opening of a receptacle of the female connector, and the flexible member configured to conform to the rigid member with a procedural barrier therebetween when the plug of the male connector is inserted into the receptacle of the female connector, thereby establishing one or more functional connections across the procedural barrier between the SPU and MPU medical devices.

2. The connection system of claim 1, wherein the flexible member is fixedly coupled to the male connector.

3. The connection system of claim 1, wherein the flexible member is removably coupled to the male connector.

4. The connection system of claim 1, wherein the flexible member is a flexible disk.

5. The connection system of claim 1, wherein the rigid member is fixedly coupled to the female connector.

6. The connection system of claim 1, wherein the rigid member is removably coupled to the female connector.

7. The connection system of claim 1, wherein the rigid member is a rigid bowl opening away from the receptacle of the female connector.

8. The connection system of claim 1, wherein the male connector is magnetically secured to the female connector upon insertion of the plug of the male connector into the receptacle of the female connector via one or more magnets in each member of the flexible member and the rigid member.

9. The connection system of claim 1, wherein the male connector is mechanically secured to the female connector upon insertion of the plug of the male connector into the receptacle of the female connector via one or more retractable protrusions protruding from the plug of the male connector and one or more corresponding recesses in an inner wall of the receptacle.

10. The connection system of claim 9, wherein the one-or-more retractable protrusions and the one-or-more corresponding recesses produce an audible click-type sound upon the insertion of the plug of the male connector into the receptacle of the female connector, thereby indicating the male connector is mechanically secured to the female connector.

11. The connection system of claim 1, wherein the male connector is adherently secured to the female connector upon insertion of the plug of the male connector into the receptacle of the female connector via an adhesive over a mating surface of each member of the flexible and rigid member.

12. The connection system of claim 1, wherein conformation of the flexible member to the rigid member forms a fluid-tight seal.

13. The connection system of claim 1, wherein the one-or-more functional connections are selected from electrical, optical, and fluid connections.

14. The connection system of claim 1, wherein the procedural barrier is a sterile drape such that the one-or-more functional connections across the procedural barrier between the SPU and MPU medical devices extend from a sterile field including the SPU medical device and a non-sterile field including the MPU medical device.

15. A method of a connection system, comprising:
inserting a male connector associated with a single patient-use (“SPU”) medical device across a procedural barrier into a female connector associated with a multiple patient-use (“MPU”) medical device, the male connector including a flexible member around the male connector with the flexible member proximal of a plug of the male connector, the female connector including a rigid member around an opening of a receptacle of the female connector, and the flexible member configured to conform to the rigid member with the procedural barrier therebetween, thereby establishing one or more functional connections across the procedural barrier between the SPU and MPU medical devices.

16. The method of claim 15, wherein the flexible member is a flexible disk fixedly or removably coupled to the male connector.

17. The method of claim 15, wherein the rigid member is a rigid bowl fixedly or removably coupled to the female connector, the rigid bowl opening away from the receptacle of the female connector.

18. The method of claim 15, wherein the male connector is magnetically, mechanically, or adherently secured to the female connector upon insertion of the plug of the male connector into the receptacle of the female connector, conformation of the flexible member to the rigid member forming a fluid-tight seal.

19. The method of claim 15, wherein the one-or-more functional connections are selected from electrical, optical, and fluid connections.

20. The method of claim 15, wherein the procedural barrier is a sterile drape such that the one-or-more functional connections across the procedural barrier between the SPU and MPU medical devices extend from a sterile field including the SPU medical device and a non-sterile field including the MPU medical device.
